(19) United States
(12) Patent Application Publication
(10) Pub. No.: US 2009/0118812 A1
(43) Pub. Date: May 7, 2009

(54) ENDOPROSTHESIS COATING

(75) Inventors: Jaydeep Y. Kokate, Plymouth, MN (US); Raed Rizq, Fridley, MN (US); Jay Rassat, Buffalo, MN (US); Derek Sutermeister, Plymouth, MN (US); Samuel Robaina, Santa Rosa, CA (US); Peter Edelman, Maple Grove, MN (US); Tom Holman, Princeton, MN (US); Michael Kuehling, Munich (DE); Yixin Xu, Newton, MA (US)

Correspondence Address: FISH & RICHARDSON PC, P.O. BOX 1022, MINNEAPOLIS, MN, 55440-1022, US

(73) Assignee: Boston Scientific Scimed, Inc., Maple Grove, MN (US)

(21) Appl. No.: 11/934,288

(22) Filed: Nov. 2, 2007

(51) Int. Cl. A61F 2/06

(52) U.S. Cl. 623/1.15; 623/1.49; 623/1.46

(57) ABSTRACT

A method includes: providing a substrate, depositing a ceramic and an extractable material onto the substrate, forming a porous structure in the ceramic by removing the extractable material, and utilizing the ceramic in an endoprosthesis. An endoprosthesis, such as a stent, including a coating formed of a ceramic and an extractable material that can be removed from the coating to form voids is also disclosed.

### TECHNICAL FIELD

This invention relates to medical devices, such as endoprostheses, and methods of making and using the same.

### BACKGROUND

The body includes various passageways including blood vessels such as arteries, and other body lumens. These passageways sometimes become occluded or weakened. For example, they can be occluded by a tumor, restricted by plaque, or weakened by an aneurysm. When this occurs, the passageway can be reopened or reinforced, or even replaced, with a medical endoprosthesis. An endoprosthesis is an artificial implant that is typically placed in a passageway or lumen in the body. Many endoprostheses are tubular members, examples of which include stents, stent-grafts, and covered stents.

Many endoprostheses can be delivered inside the body by a catheter. Typically the catheter supports a reduced-size or compacted form of the endoprosthesis as it is transported to a desired site in the body, for example the site of weakening or occlusion in a body lumen. Upon reaching the desired site the endoprosthesis is installed so that it can contact the walls of the lumen. Stent delivery is further discussed in Heath, U.S. Pat. No. 6,290,721, the entire disclosure of which is hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

It is sometimes desirable for an endoprosthesis to contain a therapeutic agent, or drug which can elute into the body fluid in a predetermined manner once the endoprosthesis is implanted.

### SUMMARY

In an aspect, the invention features a method of forming an endoprosthesis, including providing a substrate, depositing a ceramic and an extractable material onto the substrate, forming a porous structure in the ceramic by removing the extractable material, and utilizing the deposited ceramic in an endoprosthesis.

In another aspect, the invention features an endoprosthesis including a surface, and a coating over the surface, where the coating is formed of a ceramic and a void-forming salt.

In another aspect, the invention features an endoprosthesis including a surface, and a coating over the surface, where the coating is formed of a ceramic and a polymer fiber.

Embodiments may include one or more of the following features. The ceramic can be deposited onto the substrate by physical vapor deposition. The ceramic and the extractable material can be deposited simultaneously. The ceramic can be deposited without depositing the extractable material prior to simultaneously depositing the ceramic and the extractable material. The ceramic and extractable material can be deposited onto the substrate in a chamber without removing the substrate from the chamber. Multiple layers of the ceramic and the extractable material can be deposited alternately. The extractable material can be a salt selected from the group consisting of sodium halides, magnesium halides, potassium halides, and calcium halides. The extractable material can be an erodible metal. The erodible metal can be calcium, zinc, aluminum, iron, or magnesium. The extractable material can be a polymer. The polymer can be deposited by electrospinning. The extractable material can be removed by application of an organic solvent, an aqueous solution, or heat. A polymer can be deposited on the porous structure after the porous structure is formed. The polymer can include a drug. The ceramic can be selected from oxides and nitrides of iridium, zirconium, titanium, hafnium, niobium, tantalum, ruthenium, platinum, and aluminum. The ceramic can be IROX. The substrate can be the endoprosthesis body. The endoprosthesis body can be stainless steel.

Embodiments may include one or more of the following features. The coating can be about 30% or more of the salt by volume. The sale can have a domain with a width of about 10 nm to 50 nm defined by the ceramic. The domain can have a depth of about 10 nm to 500 nm. The coating can have a thickness of about 10 nm to 500 nm.

Embodiments may include one or more of the following features. The polymer fiber can be an electrospun polymer selected from polyaniline, poly-L-lactides, polyphenylene oxide, polyimides, and polysulfone. The polymer fiber can have a length of about 100 nm to 5000 nm. The polymer fiber can have a diameter of about 10 nm to 50 nm.

Embodiments may include one or more of the following advantages. An endoprosthesis, such as a stent, can be provided with a polymer coating, such as a drug eluting coating, that is strongly adhered to the stent to reduce flaking or delamination. The stent can include a porous ceramic coating, and the polymer coating can be a material that has desirable drug release characteristics but non-optimal adhesion characteristics to the ceramic material and/or stent. The adhesion can be enhanced by mechanical interlocking of the polymer and pores of the ceramic coating without modifying drug delivery or biocompatibility characteristics. Stents can be formed with a porous ceramic coating that enhance therapeutic performance. In particular, the ceramics are selected to enhance physiologic effect. Improved physiologic effects include discouraging restenosis and encouraging endothelialization. The porous structure of the ceramic coating is selected by controlling the relative amount of constituent materials in a protocoating. For example, if the protocoating is formed of half ceramic, e.g., IROX and half salt, e.g., sodium chloride, by volume, when the salt is removed, the resultant ceramic coating will have a porosity of about 50%. The protocoating can be formed by physical vapor deposition using methodologies that allow fine tuning of the composition and/or morphology characteristics and permit highly uniform, predictable coatings across a desired region of the stent.

Still further aspects, features, embodiments, and advantages follow.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIG. 3 is a cross-sectional view of a stent wall while FIG. 3A is a greatly enlarged view of the region 3A in FIG. 3.

FIGS. 4A-4C are cross-sectional views illustrating a method for forming a stent.

FIG. 5 is a schematic cross-sectional view of a magnetron sputtering system.

FIGS. 6A-6D are cross-sectional views illustrating another method for forming a stent.

FIG. 7A is an electron micrograph image of polymer fibers and FIGS. 7B-7D are cross-sectional views illustrating another method for forming a stent.

### DETAILED DESCRIPTION

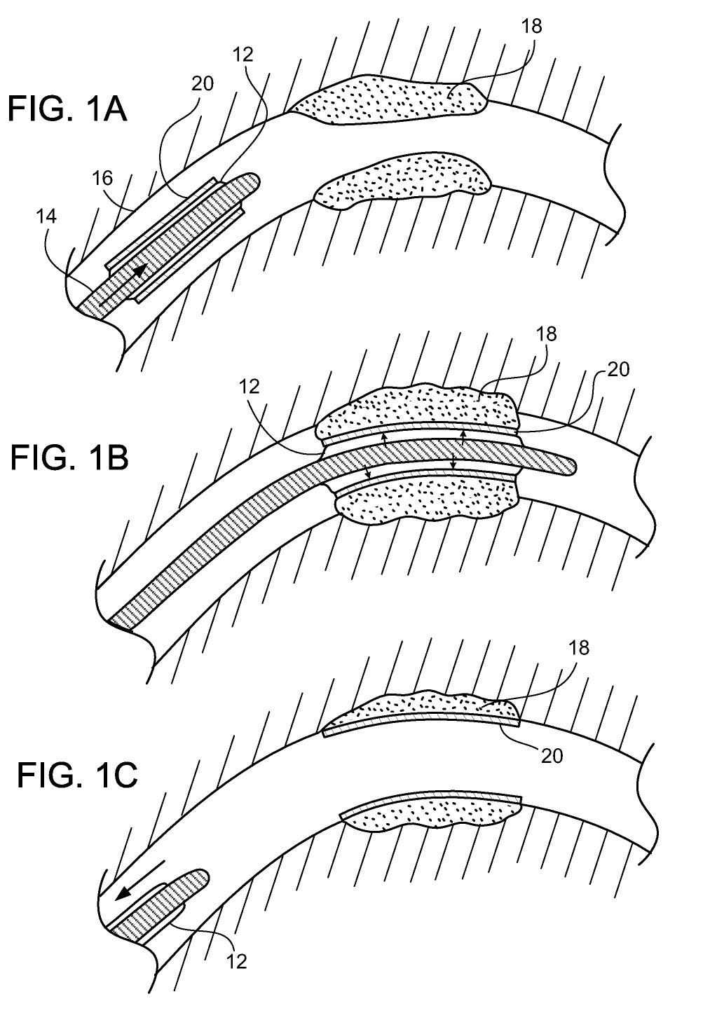

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

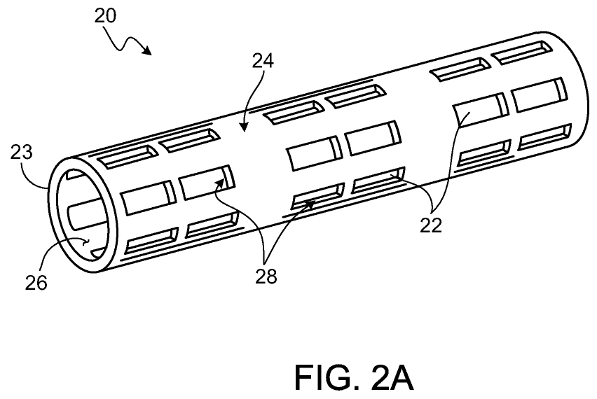

Referring to FIG. 2, the stent 20 includes a plurality of fenestrations 22 defined in a wall 23. Stent 20 includes several surface regions, including an outer, or abluminal, surface 24, an inner, adluminal, surface 26, and a plurality of cut-face surfaces 28. The stent can be balloon expandable, as illustrated above, or self-expanding stent. Examples of stents are further described in Heath '721, supra.

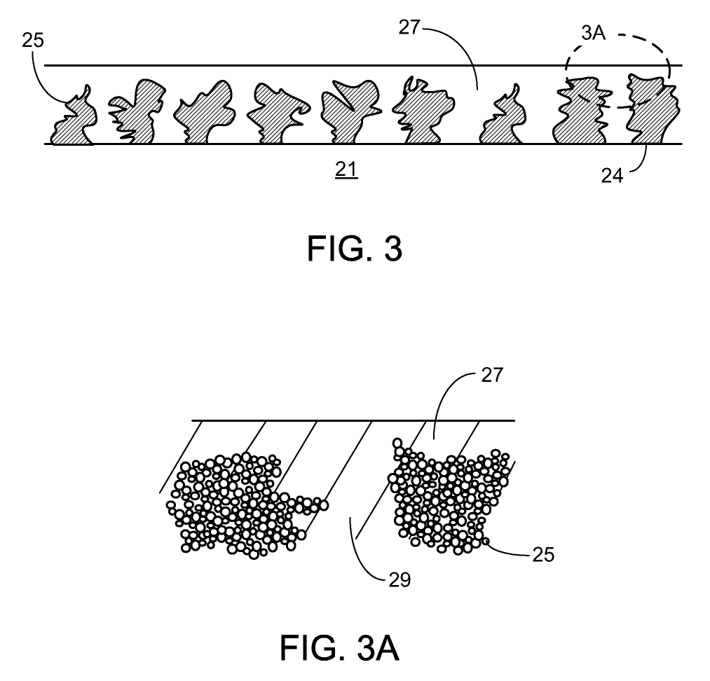

Referring to FIG. 3, a cross-sectional view, a stent wall 23 includes a stent body 21 formed, e.g. of a metal, and includes a first coating 25 formed, e.g., of a ceramic, on one side, e.g. the abluminal side 24. The first coating can be configured to have a plurality of pores or depressions in a surface. The abluminal side may also include a second coating 27, such as a polymer that includes a drug.

In embodiments, the coating 25 is formed via physical vapor deposition (“PVD”), e.g., magnetron sputtering processes, which is described in detail below. Referring particularly to FIG. 3A, an enlarged view of section 3A of FIG. 3, the ceramic coating 25 is deposited as small particles, e.g., 100 nm or less, such as 1-10 nm, and preferably smaller than the gross morphological features of the coating or layer such as depressions or pores 29 in the coating and/or rough surfaces. In embodiments, the particles bond at contact points forming a continuous coating that is an amalgamation of the particles. The second coating 27 formed, e.g., of a polymer can be applied to fill in the depressions or pores so that the polymer and the ceramic can form an interpenetrating network, which helps mechanically fix the polymer to the ceramic or enhances adhesion of the polymer to the ceramic. In embodiments, the thickness of the coating 25 is selected to be about 10 nm to 1000 nm, and the ratio of the pore volume to the total volume of solid and pores (e.g., porosity) is selected to be about 10 to 85%. The depth of pores is selected to be the same as the thickness of coating 25 or less. The diameter or average width of pores is selected to be about 10 nm to 1000 nm. In embodiments, the coating thickness can be up to about 5 μm and the average pore diameter about 10 nm-5 microns.

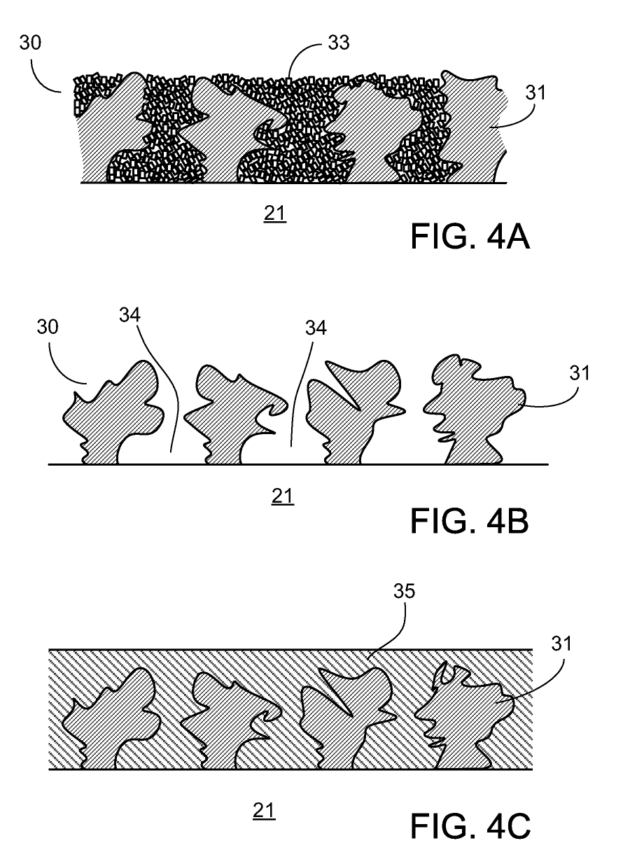

Referring to FIGS. 4A-4C, cross-sectional views of a region of a stent wall illustrate an exemplary procedure of forming a stent. Referring particularly to FIG. 4A, the stent wall includes a body 21, over which is formed a protocoating 30 of a composition including a first materials 31 (slashes) and a second material 33 (squares) on a selected side of the stent wall, such as the abluminal side. In embodiments, the composition is selected so that the first and second materials can be co-deposited onto the stent via, e.g., a PVD process, while they are separable afterwards due to their different chemical and/or physical properties. For example, referring particularly to FIG. 4B, the second material 33 can be an extractable material (e.g., a water-soluble salt) and be removed under a selected condition (e.g., soaking in water or an aqueous solution with a suitable pH value), leaving behind a porous coating formed of the first material (e.g., a water-insoluble material such as IROX) which is relatively stable. Once the extractable or elutable material is removed, depressions or pores 34 are formed where the second material used to be in the protocoating 30, increasing surface roughness and thus enhancing adhesion of a polymer to the coating. The porosity of the resultant porous coating can be selected by controlling the relative amount of the two materials deposited or composition of the protocoating, and the pore size (e.g., pore diameter, depth, and pore volume) can be selected by controlling the size of the domain in which the extractable material is defined by the more stable material of the protocoating or the crystal size of the extractable material. For example, starting with a protocoating composition of 50% of a ceramic and 50% of a salt by volume and an average salt domain size of 100 nm in diameter can result in a porous coating with a porosity of about 50% and an average pore diameter of about 100 nm. In some embodiments, the composition of the protocoating and/or the domain size of the extractable material can vary at different depth of the protocoating by, e.g., changing operating parameters of the deposition system during the deposition process. As a result, the porosity and/or pore size of the resultant coating can be variable through the depth or thickness of the coating. One application of such a configuration allows for controlling the drug release in more complex manners when the pores are loaded with a drug. Referring particularly to FIG. 4C, the pores may also provide a mechanical interlocking function as to allow formation of an interpenetrating network of a third material 35 (e.g., a polymer) and the first material 31, to enhance polymer adhesion to the stent. In embodiments, material 35 can be a drug-eluting polymer or polymer precursor, and can be applied to the first material 31 by, e.g., rolling, dipping, spraying, vapor deposition (e.g., PVD), pressing, brushing, laminating, contact printing, inkjet printing, meniscus gravure coating, sputtering, and electroplating.

In embodiments, the first material 31 is a ceramic, such as iridium oxide (“IROX”), titanium oxide (“TIOX”), TINOX (titanium oxide mixed with nickel oxide) or oxides of niobium (“Nb”), tantalum (“Ta”), all platinum group family metals, ruthenium (“Ru”), platinum, ehidium, palladium, and asminium, or mixtures thereof. Certain ceramics, e.g. oxides, can reduce restenosis through the catalytic reduction of hydrogen peroxide and other precursors to smooth muscle cell proliferation. The oxides can also encourage endothelial growth to enhance endothelialization of the stent. When a stent, is introduced into a biological environment (e.g., in vivo), one of the initial responses of the human body to the implantation of a stent, particularly into the blood vessels, is the activation of leukocytes, white blood cells which are one of the constituent elements of the circulating blood system. This activation causes an increase of reactive oxygen compound production. One of the species released in this process is hydrogen peroxide, H₂O₂, which is released by neutrophil granulocytes, which constitute one of the many types of leukocytes. The presence of H₂O₂ may increase proliferation of smooth muscle cells and compromise endothelial cell function, stimulating the expression of surface binding proteins which enhance the attachment of more inflammatory cells. A ceramic, such as IROX can catalytically reduce H₂O₂. The morphology of the ceramic can enhance the catalytic effect and reduce proliferation of smooth muscle cells. In a particular embodiment, IROX is selected to form the coating 25, which can have therapeutic benefits such as enhancing endothelialization. IROX and other ceramics are discussed further in Alt et al., U.S. Pat. No. 5,980,566 and U.S. Ser. No. 10/651,562 filed Aug. 29, 2003.

Examples of the second material 33, e.g., suitable extractable materials and proper conditions further include: a polymer such as polysulfone which can be removed by low-polar organic solvents (e.g., ketones, chlorinated hydrocarbons, and aromatic hydrocarbons), and an erodible metal such as calcium, zinc, aluminum, iron, or magnesium or soluble salts, such as halide salts, which can be removed by aqueous solution with a selected pH value. In embodiments, the polymers are thermally stable, solvent soluble polymers, such that the polymer can withstand the temperatures of a PVD process and be subsequently removed by solvent processing. Suitable polymers are described in Eur. Pol. J. 43(2) 620-7 (2007) and Polymer 45(23) 7877-85 (2004). In other embodiments, the material, e.g. a polymer, can be removed by pyrolysis. In embodiments, the polymer is a polyester, polyetherimide, polyetherimidesulfone, or an aerospace grade oligomer (e.g. polybenzoxazoles). Further polymers are described in U.S. Pat. No. 5,968,640.

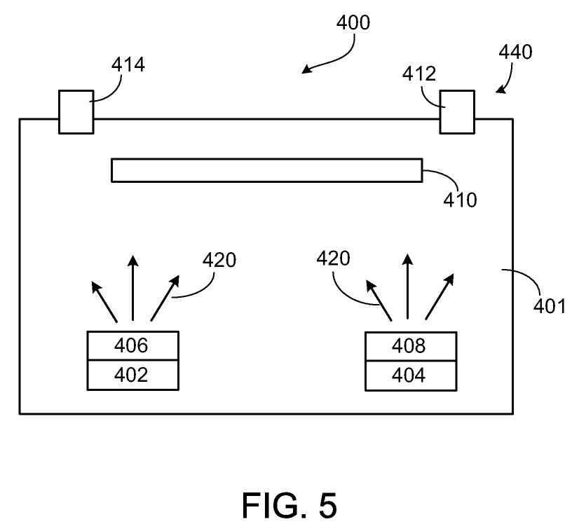

In embodiments, the first and second materials are provided over the stent by a PVD technique, such as magnetron sputtering. Referring to FIG. 5, an embodiment of a planar magnetron sputtering system is shown. System 400 includes a sputter chamber 401 having two targets 406 and 408 connected to magnetrons 402 and 404 respectively, a vacuum port 414 connected to a vacuum pump and a gas source 440 for delivering a gas, e.g., argon, to chamber 401 to generate a glow discharge plasma and cause sputtering of the targets 406 and 408. A substrate, e.g., a stent or a precursor component of a stent (“pre-stent”) 410 such as a metal tube is appropriately positioned at a distance from the targets.

In use, a power source, e.g., a negative DC voltage (not shown) is connected or applied to the target (the cathode in this circumstance) of magnitude sufficient to ionize the working gas, e.g., argon, into a plasma. The positive argon ions are attracted to the negatively charged target with sufficient energy to sputter atoms of the target material. The sputtered atoms can travel along random directions (arrows 420). Some of the sputtered atoms strike the stent and form a sputter coating thereon. The magnetron, usually positioned in back of the target, can create a magnetic field adjacent and lying principally parallel to the target. The magnetic field traps electrons close to the surface of the target. The electrons follow helical paths around the magnetic field lines undergoing more ionizing collisions with neutral argon gas near the target surface than would otherwise occur. The extra argon ions created as a result of these collisions leads to a higher deposition rate. It also means that the plasma can be sustained at a lower pressure. Charge build-up on insulating targets can be avoided with the use of radio frequency (“RF”) sputtering where the sign of the anode-cathode bias is varied at a high rate. In some embodiments, for reactive sputtering, other gases such as oxygen or nitrogen can be fed into the sputter chamber in addition to argon, to produce oxides or nitrides films.

In embodiments, targets can connect to a common power source or separate power supplies. In embodiment, the targets 406 and 408 may be sputtered simultaneously. In certain embodiments, the target 406 is a ceramic, such as iridium oxide (“IROX”), or a mixture of a metal and a ceramic, such as a mixture of iridium and IROX; while the target 408 is a salt, such as halides of sodium, magnesium, calcium or potassium. In certain embodiments, the target 406 is a ceramic or a mixture of a metal and a ceramic while the target 408 is a polymer, e.g., thermally stable or heat-resistant polymers, such as polyphenylene oxide (PPO), polyimides, polysulfone, and polyamides. In other embodiments, only one target is sputtered and the target is a mixture of a ceramic and a salt or a mixture of a ceramic and a polymer. In embodiments, a polymer coating can be deposited onto the stent using polymer particles of desired size and shape, and the ceramic coating subsequently deposited into the polymer.

The operating parameters of the deposition system are selected to tune the morphology and/or composition of the sputter coating, e.g., a mixture of a ceramic and a salt or polymer. The composition of the deposited material is selected by controlling the connection of the target materials to an applied high electric potential, usually a negative potential, or by controlling the exposure of the target materials to working plasma. For example, to deposit pure ceramic or pure salt, only the ceramic material or salt is exposed to plasma; to deposit a composite layer of ceramic and salt, both materials are exposed simultaneously or alternately exposed in rapid succession. In particular, the power, total pressure, oxygen/argon ratio and sputter time are controlled during the deposition process. In embodiments, the power is within about 340 to 700 watts, e.g. about 400 to 600 watts and the total pressure is about 10 to 30 mTorr. In other embodiments the power is about 100 to 350 watts, e.g. about 150 to 300 watts, and the total pressure is about 1 to 10 mTorr, e.g. about 2 to 6 mTorr. The oxygen/argon ratio is in the range of about 10 to 90%. The deposition time controls the thickness of the ceramic and/or the salt. In embodiments, the deposition time is about 0.5 to 10 minutes, e.g. about 1 to 3 minutes. The overall thickness of the sputter coating is about 50-500 nm, e.g. about 100 to 300 nm. The oxygen content is increased at higher power, higher total pressure and high oxygen to argon ratios. The substrate temperature is also controlled. The temperature of the substrate is between 25 to 300° C. during deposition. Substrate temperature can be controlled by mounting the substrate on a heating element.

Other sputtering techniques or systems can be used to form a stent coating. For example, an inverted cylindrical physical vapor deposition arrangement may include a cathode in the shape of a cylinder on the luminal side of which resides a target, such as a ceramic (e.g. IROX) or a ceramic precursor metal (e.g. Ir). A stent (or precursor component of a stent) is usually disposed in the center of the cylinder. The cylinder includes a gas, such as argon and oxygen. A plasma formed in the cylinder accelerates charged species toward the target. Target material is sputtered from the target and is deposited onto the stent.

Physical vapor deposition is described further in SVC: Society of Vacuum Coatings: C-103, An Introduction to Physical Vapor Deposition (PVD) Processes and C-248—Sputter Deposition in Manufacturing, available from SVC 71 Pinion Hill, Nebr., Albequeque, N. Mex. 87122-6726. A suitable cathode system is the Model 514, available from Isoflux, Inc., Rochester, N.Y. In other embodiments, pulsed laser deposition (“PLD”) is utilized to form a coating. PLD is described in co-pending applications U.S. application Ser. No. 11/752,735 and U.S. application Ser. No. 11/752,772, filed concurrently. In particular embodiments, the ceramic has a selected morphology as described in U.S. application Ser. No. 11/752,735 and U.S. application Ser. No. 11/752,772. Formation of IROX is also described in Cho et al., Jpn. J. Appl. Phys. 36(I) 3B: 1722-1727 (1997), and Wessling et al., J. Micromech. Microeng. 16:5142-5148 (2006).

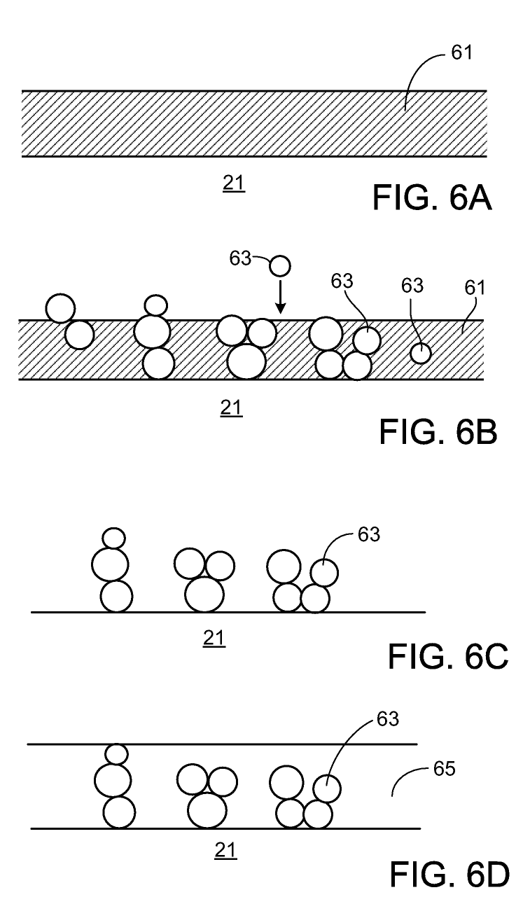

Referring to FIGS. 6A-6D, another exemplary procedure of forming a stent is illustrated. Referring particularly to FIG. 6A, a cross-sectional view of a region of a stent wall, the stent wall includes a body 21 over which is pre-deposited a polymeric coating 61. The polymer coating 61 can be formed by, e.g., rolling, dipping, spraying, vapor deposition (e.g., PVD), pressing, brushing, or laminating. Since the polymer is pre-deposited, heat sensitive polymers unsuitable for sputtering can also be used and applied by, e.g., dipping, spraying or rolling, or printing techniques as described above. The polymer coating can be used as a sacrificial template. In some embodiments, a ceramic coating can be deposited onto the stent before the polymeric coating. In still some embodiments, the polymer coating can be applied with another extractable material, e.g., a salt, to the stent before sputtering the ceramic material.

Referring particularly to FIG. 6B, a ceramic is deposited over or into the polymer coating 61 by, e.g., sputtering as discussed above. The ceramic is deposited as small particles 63. The particles may be adhered on top of the polymer or on top of the stent body by penetrating or damaging the polymer due to their different kinetic energies. Some particles may bond at contact points forming a relatively continuous coating that is an amalgamation of the particles adhered to the stent. The polymer coating 61 can act like a buffer that reduces the kinetic energies of the sputtered particles and thus a less dense coating or a more porous structure can be formed compared to those formed without the polymer coating. In some embodiments, a second polymer coating can be applied to the ceramic-polymer mixture and another round of ceramic deposition can be carried out using e.g., the same ceramic or a different ceramic, in similar manners as illustrated in FIGS. 6A and 6B. The ceramic and polymer can be alternately deposited to form multiple layers until derisible configurations and functions of the surface are achieve, e.g., surface roughness to enhance polymer adhesion, therapeutic effect of the ceramic to enhance endothelial cell growth, and predetermined porous structures to obtain desired drug release profiles. Referring particularly to FIG. 6C, when the polymer coating is removed by, e.g., an organic solvent or heat treatment such as burning, the particles unattached to the others or the stent may be removed as well, leaving behind a continuous ceramic coating with a porous structure on the stent. Referring particularly to FIG. 6D, a drug-eluting polymer 65 is then provided over the ceramic with enhanced adhesion due to the porous structure of the ceramic coating.

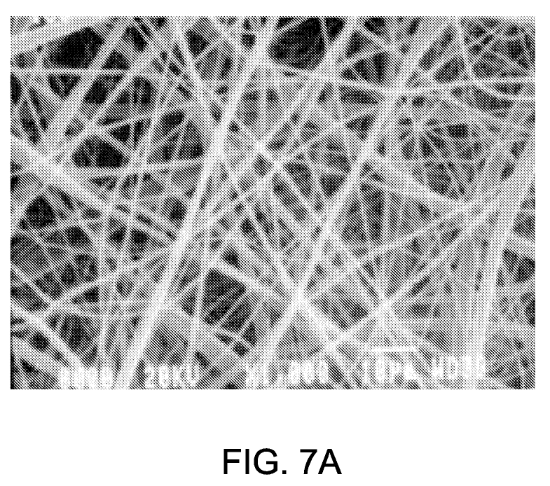

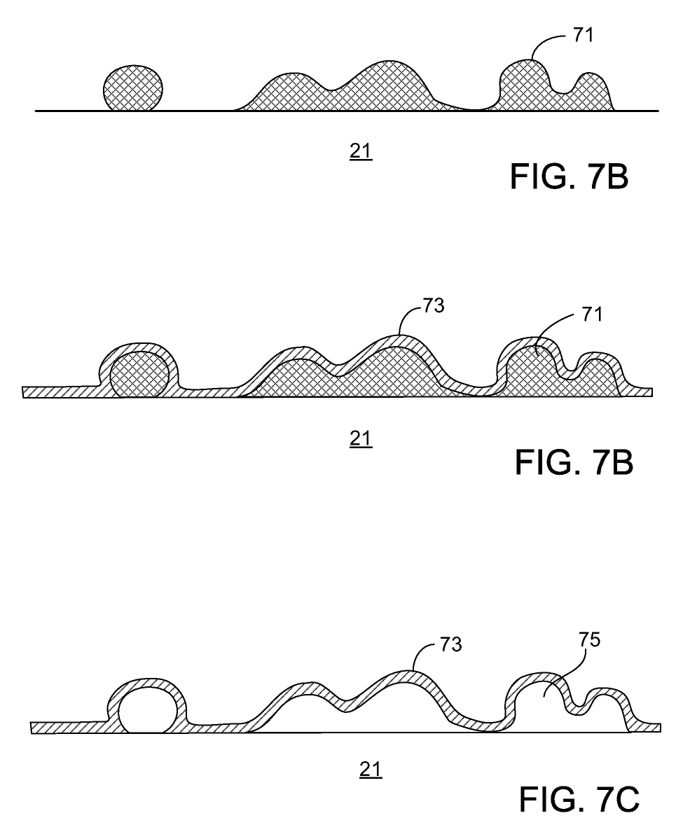

Referring to FIGS. 7A-7D, in a particular embodiment, a pre-deposited polymer coating can be formed by electrospinning polymer fibers to form a network over the stent surfaces, e.g., abluminal surfaces. Referring particularly to FIG. 7A, a scanning electron microscopy picture shows the fiber network formed of poly-L-lactides (PLLA). In embodiments, the diameter, length, and density of the fibers can be controlled by, e.g., concentration of the polymer in a polymer suspension for electrospinning, the applied electric potential, and the flow rate of the suspension. In some embodiments, a ceramic e.g., IROX layer may be deposited on the stent prior to the polymer fibers. Exemplary polymers include polyaniline, and poly-L-lactides (PLLA). FIG. 7B is a cross-sectional view of a region of a stent wall. The stent wall includes a body 21 over which is a polymer fiber network 71 formed by electrospinning. Referring particularly to FIG. 7C, a ceramic 73, e.g., IROX, is deposited over the polymer network 71 by, e.g., sputtering as discussed above. The polymer fibers can function as a sacrificial template. Accordingly, the gross morphological features (e.g., depressions, surface roughness) of the ceramic coating 73 that overlies the polymer template 71 can be controlled by selecting the structure of the fiber network, e.g., by controlling the density of the fibers, the diameter and length of the fibers. Referring particularly to FIG. 7D, when the polymer template is removed by, e.g., an organic solvent or heat treatment such as burning, the ceramic coating 73 remains on the stent with the same morphological features as shown in FIG. 7C and tunnels 75 of the shape of the polymer fibers underneath the ceramic. The gross morphological features can enhance the adhesion of polymers to the ceramic coating. In some embodiments, the tunnels can be used as drug reservoirs. Polymer electrospinning is discussed in U.S. Ser. No. 11/694,436, filed Mar. 30, 2007 [Attorney Docket No. 10527-068001], Zeng et al., Journal of Controlled Release 92 (2003) 227-231, and Journal of Industrial Textiles 36:4 (2007) 311-327.

In embodiments, ceramic is adhered only on the abluminal surface of the stent. This construction may be accomplished by, e.g. coating the stent before forming the fenestrations. In other embodiments, ceramic is adhered only on abluminal and cutface surfaces of the stent. This construction may be accomplished by, e.g., coating a stent containing a mandrel, which shields the luminal surfaces. Masks can be used to shield portions of the stent. In embodiments, the stent metal can be stainless steel, chrome, nickel, cobalt, tantalum, superelastic alloys such as nitinol, cobalt chromium, MP35N, and other metals. Suitable stent materials and stent designs are described in Heath '721, supra. In embodiments, the morphology and composition of the ceramic are selected to enhance adhesion to a particular metal. For example, in embodiments, the ceramic is deposited directly onto the metal surface of a stent body, e.g. a stainless steel, without the presence of an intermediate metal layer. In other embodiments, a layer of metal common to the ceramic is deposited onto the stent body before deposition to the ceramic. For example, a layer of iridium may be deposited onto the stent body, followed by deposition of IROX onto the iridium layer. Other suitable ceramics include metal oxides and nitrides, such as of iridium, zirconium, titanium, hafnium, niobium, tantalum, ruthenium, platinum and aluminum. The ceramic can be crystalline, partly crystalline or amorphous. The ceramic can be formed entirely of inorganic materials or a blend of inorganic and organic material (e.g. a polymer).

Suitable drug eluting polymers may be hydrophilic or hydrophobic, and may be selected, without limitation, from polymers including, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenerated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present disclosure. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the suitable polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyiocyanates such that the devices become instantly lubricious when exposed to body fluids. Another suitable polymer is a copolymer of polylactic acid and polycaprolactone. Suitable polymers are discussed in U.S. Publication No. 2006/0038027.

The polymer is preferably capable of absorbing a substantial amount of drug solution. When applied as a coating on a medical device in accordance with the present disclosure, the dry polymer is typically on the order of from about 1 to about 50 microns thick. In the case of a balloon catheter, the thickness is preferably about 1 to 10 microns thick, and more preferably about 2 to 5 microns. Very thin polymer coatings, e.g., of about 0.2-0.3 microns and much thicker coatings, e.g., more than 10 microns, are also possible. It is also within the scope of the present disclosure to apply multiple layers of polymer coating onto a medical device. Such multiple layers are of the same or different polymer materials.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application No. 2005/019265A.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721).

In embodiments, the ceramic layer and drug-eluting polymer layer are provided only on the abluminal surface, as illustrated. In other embodiments, these elements are provided as well or only on the adluminal surface and/or cut-face surfaces.

All publications, patent applications, patents, and other references mentioned herein are incorporated by reference herein in their entirety.

Still further embodiments are in the following claims

What is claimed is:

1. A method of forming an endoprosthesis, comprising:
providing a substrate,
depositing a ceramic and an extractable material onto the substrate,
forming a porous structure in the ceramic by removing the extractable material, and
utilizing the ceramic in an endoprosthesis.

2. The method of claim 1 wherein the ceramic is deposited onto the substrate by physical vapor deposition.

3. The method of claim 1 comprising simultaneously depositing the ceramic and extractable material.

4. The method of claim 3 further comprising depositing the ceramic without depositing extractable material prior to simultaneously depositing the ceramic and the extractable material.

5. The method of claim 1 comprising depositing the ceramic and extractable material onto the substrate in a chamber without removing the substrate from the chamber.

6. The method of claim 1 comprising alternately depositing multiple layers of the ceramic and the extractable material.

7. The method of claim 1 wherein the extractable material is a salt selected from the group consist of sodium halides, magnesium halides, potassium halides and calcium halides.

8. The method of claim 1 wherein the extractable material is an erodible metal.

9. The method of claim 8 wherein the erodible metal is calcium, zinc, aluminum, iron, or magnesium.

10. The method of claim 1 wherein the extractable material is a polymer.

11. The method of claim 10 comprising depositing the polymer by electrospinning.

12. The method of claim 1 comprising removing the extractable material by application of an organic solvent, an aqueous solution, or heat.

13. The method of claim 1 further comprising depositing a polymer on the porous structure after the porous structure is formed.

14. The method of claim 13 wherein the polymer includes a drug.

15. The method of claim 1 wherein the ceramic is selected from oxides and nitrides of iridium, zirconium, titanium, hafnium, niobium, tantalum, ruthenium, platinum and aluminum.

16. The method of claim 15 wherein the ceramic is IROX.

17. The method of claim 1 wherein the substrate is the endoprosthesis body.

18. The method of claim 17 wherein the endoprosthesis body is stainless steel.

19. An endoprosthesis, comprising:
a surface, and
a coating over the surface, wherein the coating is formed of a ceramic and a void-forming salt.

20. The endoprosthesis of claim 19 wherein the coating has about 30% or more of the salt by volume.

21. The endoprosthesis of claim 19 wherein the salt has a domain with a width of about 10 nm to 50 nm defined by the ceramic.

22. The endoprosthesis of claim 21 wherein the domain has a depth of about 10 nm to 500 nm.

23. The endoprosthesis of claim 19 wherein the coating has a thickness of about 10 nm to 500 nm.

24. An endoprosthesis, comprising:
a surface, and
a coating over the surface, wherein the coating is formed of a ceramic and a polymer fiber.

25. The endoprosthesis of claim 24 wherein the polymer fiber is an electrospun polymer selected from polyaniline, poly-L-lactides, polyphenylene oxide, polyimides, and polysulfone.

26. The endoprosthesis of claim 24 wherein the polymer fiber has a length of about 100 nm to 5000 nm.

27. The endoprosthesis of claim 24 wherein the polymer fiber has a diameter of about 10 nm to 50 nm.
